(19) United States
(12) Patent Application Publication
(10) Pub. No.: US 2007/0244376 A1
(43) Pub. Date: Oct. 18, 2007

(54) PHYSIOLOGICAL SIGNAL APPARATUS WITH DIGITAL REAL TIME CALIBRATION

(76) Inventor: Wei-Kung Wang, Taipei (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/736,223

(22) Filed: Apr. 17, 2007

(30) Foreign Application Priority Data
Apr. 18, 2006 (TW) 095113936

(51) Int. Cl. A61B 5/00

(52) U.S. Cl. 600/310; 600/336; 600/300; 600/595

(57) ABSTRACT

A digital vital sign apparatus with real time calibration is provided which comprises a tissue adaptor, several physiological signal measurement devices with an ability of movement detection, and a physiological analyzer. The present invention provides wrapping a tissue like finger for testing, palm or wrist with elastic membrane to buffer the noise. Electro-optical, pressure, electrical sensor, motion sensor and so on are sued to detect the physiological signals as parameters. These parameters can be personal and time-dependent. The detectors for the physiological signals work at time when there is no movement to give stable signal.

### FIELD OF THE INVENTION

The present invention is related to a therapeutic apparatus, more specifically, to an apparatus for monitoring physiological signals.

### BACKGROUND OF THE INVENTION

Physiological measurements need steady signal so that analyses can be conducted. Most apparatus or monitors therefore use all-or-none vital signals such as heartbeat, breath, blood pressure or pulse etc. to determine if a subject is dead or close to death based on the vital signals. More subtle signals such as blood oxygen or diastolic and systolic pressure need more work in order to fix a probe or transducer, and fixing these probes or transducers is very difficult.

Subjects in motion always make these apparatus or monitors inoperable. In the present invention, we introduce some new physiological parameters, as well as a movement detecting algorithm which will first classify the condition of the person under surveillance as still (no movement), chaotic, or normal movement to solve the above-mentioned problems previously met in the prior art.

### SUMMARY OF THE INVENTION

The present invention provides a digital vital sign apparatus with real time calibration. The digital vital signal apparatus comprises a tissue adaptor, several physiological signal measurement devices with a ability of movement detection, and a physiological analyzer, wherein said devices are fixed into a body part of a user by said tissue adaptor to measure a signal of movement, and said analyzer makes analysis on said signal to tell if said user is in normal movement or chaotic physiological phenomena or no movement.

Other features and advantages of the present invention will be apparent from the accompanying drawings and from the detailed description that follows below.

The present invention also provides ways of adjusting the parameter for safety evaluation based on every precedent evaluation that is deemed safe. According to the present invention, a real-time calibration scheme greatly increases the quantitatively analytic power of this system. The unique figure of the present is the motion detector that will distinguish movement into chaotic physiological events and ordinary movements that implies save. The selection of sensor can be user-dependent. It depends on the specific physiological response, and specific physiological parameter of this user. It is especially useful to select parameters which are deemed normal at the moment of use from the user. The parameters can be spectrum of transmitted light which it changes with time, and the pulse wave which is also changes with time.

### BRIEF DESCRIPTION OF THE DRAWING

FIG. 1 shows a backside of a palm with the apparatus according to one embodiment of the present invention.

FIG. 2 shows a front side of a palm with the apparatus according to one embodiment of the present invention.

### DETAILED DESCRIPTION OF THE INVENTION

Only when the movement-detecting algorithm transmits “a signal of no normal movement,” will the physiological measurements begin, while the chaotic conditions will be verified by other physiological measurements.

If the “normal movement” signal is transmitted, which actually shows that a person is safe at this very moment, we can keep the movement detection on, and make next assessment a few seconds (say 20 seconds) later.

If there is no movement signal detected, it implies that the subject is quiet or still, and this comprises two possibilities: the subject is healthy and quiet or the subject is dying. We need to activate other physiological devices to distinguish between these two states. The possible devices comprise traditional EKG, and breath detector. They may also comprise pulse analysis, tissue transparency analysis, and oxygen debt analysis or blood compositions by mold-in method etc.

If the subject is suspected of being in the chaotic physiological condition, that is defined by some repeated small movements, and these can be picked up by the acceleration detection or the baseline movement of the physiological measurement device.

For these chaotic physiological conditions, the baseline of the measurement may drift with time, which is small comparing to the total signal, and we may first use frequency analysis to make detections if this is a repeated small signal. It may then be confirmed by some noise resistance parameters such as physical properties of the skin which comprise skin moisture, skin temperature, electrical property (conductivity, impedance), smoothness (goose pimples), light scattering at skin surface, and skin chemical properties, such as water content, pH, or NH₃, or Cl⁻, or Na⁺ concentration. Tissue optical properties use total intensity such as oxygen debt, transparency at specific wavelength, microcirculation indicators etc. These parameters giving large signals and small movement can be considered as perturbation that will not cause difficulty in judging if the subject is entering chaotic physiological conditions, as mentioned. After detecting stillness, there are a few special properties for some physiological parameters.

In the present invention, we give these parameters with grades to measure the health deterioration, for example the oxygen debt parameter which measures the oxygen depth in our tissue. The traditional pulse oxygen meter uses a pulse signal only, and compare the oxy-hemoglobin signal with the deoxy-hemoglobin signal; therefore, it can measure only the sudden drop of the oxygen content, the reading will not drop further and the parameter becomes useless.

The oxygen debt parameter according to the present invention can use both pulse and total signals. It uses the initial reading and defines it as 100%, and then the reading can keep going lower and lower, which suggests that the oxygen debt become deeper and deeper. There is not an all-or-none response; it gives grade or depth into oxygen debt. It can monitor not only the sudden change such as death, but also it can be used as a monitor for the health condition of ordinary persons.

Tissues are composed of arteries, veins, capillaries, cells, and interstices. When oxygen supply is insufficient, the first response that is the deoxy-hemoglobin will increase, when there is not enough deoxy-hemoglobin movement away from the capillary, the CO₂ cannot be moved with the deoxy-hemoglobin. When the CO₂ is adsorbed by H₂O to form H₂CO₃=H⁺+HCO₃⁻, these two ions will increase the osmotic pressure in the interstitial tissue, and therefore it induce an edema therein.

Using this physiological phenomenon, we can monitor the water edema signal in the interstitial tissue as an additional signal of the oxygen debt. This renders a grade in all level of oxygen debt.

It can therefore be used to check the effect of medicinal, cosmetic, rehabilitation, physical, or therapeutic treatment or any act that may improve blood circulation or oxygen content in the tissue.

Measuring the oxygen debt is very important in athletes as well as in health check-ups, if the athlete can run 100 m within a certain time limit, while the change of oxygen debt is quite small, this indicates a good athlete with great potential. The same applies to swimmers, football player and so on. For ordinary check-up, we may challenge the person with low oxygen air (<21%); if the oxygen debt does not change, that indicates the person has good heart-lung function. The lower oxygen content he/she can tolerate the better heart and lung function he has. If the person is in oxygen debt, we may let him/her breathe air with high oxygen content (>21%). By measuring the oxygen content in the air he/she breathes thus we can assess how deep he/she is in oxygen debt.

Actually any act that may change oxygen content in the tissue can be used to test the tolerance of the subject and can be used to evaluate the health condition of the subject.

For all electro-optical studies on the tissues, 530 nm and the surrounding wavelength, 800 nm and the surrounding wavelength, are very important; around these bands, the deoxy-hemoglobin and oxy-hemoglobin have a similar absorption coefficient. Equally important bands are 660 nm and the surrounding wavelength, and 940 nm and the surrounding wavelength. Both have large absorption differences for deoxy-hemoglobin and oxy-hemoglobin. These four bands become the most important wavelength for tissue transparency, oxygen debt and all electro-optical studies. One to four wavelengths are selected. Ultra violet or infrared wavelength that is useful for detecting other blood ingredients can also be used. We can create all kinds of devices for physiological measurement. When two or more signals are used, we can use a mold-in method to find out the concentration in the blood or in the whole tissue, depending on if we choose a signal from the artery or a signal from the whole tissue.

The pulse analysis can be another parameter to pursue. The pulse analysis uses amplitude and phase or difference harmonics of the pulse to detect the problems in each corresponding meridian and organ. The CV (coefficient of variance) pulse amplitude and phase can be additional parameters; the C0: 0 harmonic can especially be used to assess the condition of the circulatory system, and CV of C1 can be used to assess how close the subject is to death. For any two time-dependent signals, no matter if they are optical, electrical property, transparency, pressure pulse shape, and blood flow, the following algorithm can be used to analyze them. For any two signals of time dependent A(t) and B(t), A and B represent any physiological parameter, and we may apply mold-in algorithm A(t)/B(t)=k, where k is a complex number to adapt the phase difference in these two parameters, and when A(t)≅ electrical potential B(t)≅current, the k is impedance, when A(t) is blood pressure, B(t) is blood flow, k is microcirculation impedance, etc.

The amplitude and phase relationship can be applied to any two parameters. For the pulse shape detection, it can be the pressure sensor, which measures the pressure change at one specific spot on the artery. It may also measure the volume of blood by an electro-optical method as the change of light absorption according to time, because the volume of blood is correlated with the pressure inside the artery. We may thus figure out the pressure change according to time using the mold-in method. If the subject has normal movement, this vital indicator will judge the condition of the subject as safe. Although there is a tissue adapter made by buffering materials such as an elastic membrane, soft pad etc., to fix the sensors on the body and buffer the effect of the movement, each movement can still introduce a position change for the sensors. According to the present invention, after every movement detection or detection of stillness that activates the physiological measuring device, the parameters should be reviewed according to this reading. The apparatus according to the present invention therefore has all the proper parameter for each indicator for this new position.

To focus on the advantage of each indicator, we may select the physiological devices, according to past experience, for example, hyperglycemia symptoms may be different for different subjects, so we may select an electrical skin sensor, if the subject is used to sweating a lot during hyperglycemia. If the subject is used to having pounding heart, we may choose pulse analysis. If the subject is used to having cold hands and feet, we may choose the skin temperature detector, and so on.

Each user may also have different parameters in a normal state. To improve the sensitivity of this indicator, each physiological measuring device should be calibrated at the start of using the device, that is, to make one measurement after the sensors are put on, and then use these readings as the calibration to adjust all the parameters. All these calibrations are constantly adjusted to the present condition of the user and the current position of the sensor on the user is defined as real time calibration.

To detect the movement of the user, acceleration detection is one of the choices; every movement must be associated with acceleration to start the move, and then deceleration to stop the movement. This acceleration or deceleration can also affect the sensor, and due to the elastic tissue adapter, the sensor's movement will be much slowed; however, it will still introduce some relative movement between the sensor and the user who wear it. This can be distinguished by the baseline drift from the real signal. Several consecutive readings are required. If the slope change is faster than a threshold, it is a movement. If the reading keeps going in one direction, in a slower path for a defined time, it is a real signal. From the analysis of the sensor's signal, we can distinguish between the baseline drift and the real signal. It is possible to use the sensor itself as a movement detector, especially those sensors with a noise-resistance property. In this disclosure, the noise resistance properties comprise oxygen debt, tissue transparency, skin impedance, and skin temperature. These apparatus can work both as physiological signal detectors and movement detectors, and can therefore work alone as vital signal indicators as well as vital signal monitors or work together with other sensors to work as physiological signal detectors as well as movement detectors. According the present invention, the apparatus with real time calibration can work in all conditions as a monitor. It will sound an alarm whenever it detects danger; these signals comprise chaotic physiological conditions, elevated CO of pulse (zero harmonic), elevated CV (coefficient of vaccine) of C1 (first harmonic) of pulse, a sudden increase or fall below a threshold of oxygen debt, weak pulse, and low skin temperature.

It may set an alarm for the user first; this alarm can be a buzzing sound, ringing, vibration or mild electrical stimulation etc., to wake the user up. At the same time, the relief action may begin, to give well designed emergency treatment, which is calculated according to the user's need before the onset of the monitor.

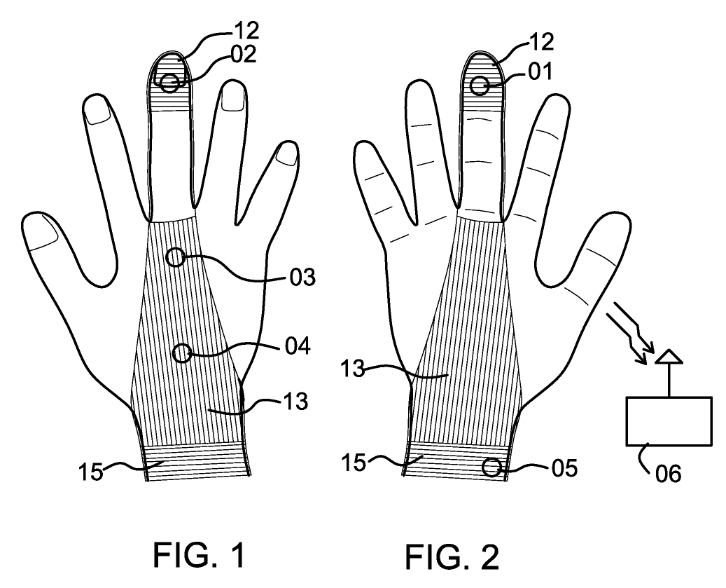

FIGS. 1 and 2 are schematic diagrams of a digital vital sign apparatus with real time calibration according to one embodiment of the present invention.

In FIG. 1, an elastic material wraps an entire middle finger and most of the palm and the whole wrist. A light source element O₂ of optical sensor wrapped in an opaque portion with horizontal lines 12 which is a finger cap in harder material. A temperature sensor 03 and a movement signal detector 04 are wrapped in an opaque portion with vertical lines 13 in elastic material.

In FIG. 2, an electro-optical sensor element for detecting light intensity 01 is wrapped in an opaque portion with horizontal lines 12. A pressure or electro-optical sensor 05 for detecting pulses in radial arteries is wrapped in a portion with horizontal lines by elastic material 15, and fastened on a wrist. A signal receiver 06 comprises a signal analyzer and an alarm device. The signal receiver 06 is able to receive the detected signals in either wire or wireless from the sensors 01, 03, 04 and 05, and then the signals are analyzed. In case the detected signals shows a life in danger, then an alarm is sound. The alarm can be wirelessly transmitted to a user, a nurse or a guardian.

### EXAMPLE

For a user who had hyperglycemia symptoms, some glucose was automatically injected into the blood stream through intravenous injection or tube feed. For a user who had asthma, a drug was automatically delivered through injection or into the air to be inhaled. For a user who snoozed, a respirator or electrical acupuncture started operating to sooth the symptom. These help-in-demand concepts were used in many more mild medical or physiological problems.

After 20 seconds of the self-alarm, if the user did not wake up, the alarm went automatically to the helper or medical staff through ring, voice, vibration or mild electrical stimulator to call for help. The used physiological signals according to the present invention were digital signal with grade.

What is claimed is:

1. A digital vital sign apparatus with real time calibration comprising
a tissue adaptor;
several physiological signal measurement devices with a ability of movement detection, and
a physiological analyzer,
wherein said devices are fixed into a body part of a user by said tissue adaptor to measure a signal of movement, and said analyzer makes analysis on said signal to tell if said user is in normal movement or chaotic physiological phenomena or no movement.

2. An apparatus as claimed in claim 1, wherein said physiological signal measurement devices with movement detecting ability comprise an accelerating detection, or a movement baseline of physiological signal detection.

3. An apparatus as claimed in claim 2, wherein said physiological analyzer is activated when there is no normal movement detected.

4. An apparatus as claimed in claim 3, as said movement detection judge as chaotic physiological phenomena, the physiological analyzer activates those noise resistant signals to determine if said user is in chaotic physiological conditions.

5. An apparatus as claimed in claim 4, wherein said chaotic physiological condition comprises asthma, hypoglycemia, snoring, complications of hypertension, heart disease, twitching and spasms.

6. An apparatus as claimed in claim 3, wherein said noise resistant signals comprise skin signal, physical properties, skin chemical properties, oxygen debt, tissue optical properties or microcirculation.

7. An apparatus as claimed in claim 2, wherein said analysis made by said physiological analyzer further comprises blood sugar, pulse, other blood components, tissue optical spectrum or a combination thereof.

8. An apparatus as claimed in claim 1, wherein said each physiological signal measurement devices are selected by said user's need.

9. An apparatus as claim in claim 1, wherein said parameters for said analysis are set by said user's need.

10. An apparatus as claimed in claim 1, wherein said apparatus is used as a monitor.

11. An apparatus as claimed in claim 1, wherein said apparatus is used on a sleeping or unconscious user.

12. An apparatus as claimed in claim 1, wherein said signal analysis set new parameters according to each of safe measurements.

13. An apparatus as claimed in claim 1, wherein each of said physiological signals is analyzed by several subsequent signals at different time.

14. An apparatus as claimed in claim 1, wherein each of said physiological signals is analyzed by the change in the signal with time.

15. An apparatus as claimed in claim 1, wherein said parameters are set at a first use.

16. An apparatus as claimed in claim 1, wherein said physiological signal starts to be analyzed when there is no movement signal detected.

17. An apparatus as claimed in claim 16, wherein new parameters are set at each of measurements.

18. An apparatus as claimed in claim 1, wherein said signal analysis comprises frequency analysis.

19. An apparatus as claimed in claim 1, wherein said signal comprises a variation part.

20. An apparatus as claimed in claim 1, wherein said signal comprises a total signal.

21. An apparatus as claimed in claim 1, wherein said signal analysis comprises amplitude, phase, CV of amplitude or CV of phase.

22. An apparatus as claimed in claim 1, wherein said signal comprises an optical signal.

23. An apparatus as claimed in claim 22, wherein said optical signal comprises 940 nm and the nearby wavelength, or 530 nm and the nearby wavelength, or 660 nm and the nearby wavelength, or 800 nm and the nearby wavelength.

24. An apparatus as claimed in claim 1, further comprising an alarm system which initiates an alarm at a preset condition.

25. An apparatus as claimed in claim 24, wherein said alarm system comprises a relief device.

26. An apparatus as claimed in claim 25, wherein said alarm system comprises a self-alarm or a call out alarm.

27. An apparatus as claimed in claim 3, wherein said signals comprise A(t), B(t), both being functions of time and A(t)/B(t)=k, where k comprise complex number and k is a parameter.

28. An apparatus as claimed in claim 6, wherein said oxygen debt comprises a property related to the oxygen content in the tissue.

29. An apparatus as claimed in claim 28, wherein said property comprises a property of water or hemoglobin.

30. An apparatus as claimed in claim 24, wherein said preset condition is at a first measurement determined as safe.

31. An apparatus as claimed in claim 24, wherein said present condition is reset according to every measurement determined as safe.

32. An apparatus as claimed in claim 29, wherein between two subsequent measurements said user goes through treatments that comprise rehabilitation, cosmetic or medical treatment, physical therapy, breathing air with elevated oxygen content or any act that may improve blood circulation or oxygen content in the tissue.

33. An apparatus as claimed in claim 29, wherein between two measurements said user goes through treatments that comprise breathing air with lower oxygen content, exercise, or any act that may lower the blood circulation or reduce oxygen content in said tissues.

34. An apparatus as claimed in claim 1, wherein said user is considered as safe when a normal movement signal is detected.

35. An apparatus as claimed in claim 1, wherein said tissue adaptor comprises buffering materials.
